(19) United States
(12) Patent Application Publication
(10) Pub. No.: US 2003/0119209 A1
(43) Pub. Date: Jun. 26, 2003

(54) Diagnostic methods and devices

(76) Inventors: Rosann Marie Kaylor, Cumming, GA (US); Chibueze O. Chidebelu-Eze, Atlanta, GA (US); Laura Michelle Folkenberg, Alpharetta, GA (US); Bruce Scott Williamson, Alpharetta, GA (US)

Correspondence Address: KIMBERLY-CLARK WORLDWIDE, INC., 401 NORTH LAKE STREET, NEENAH, WI, 54956

(21) Appl. No.: 10/026,314

(22) Filed: Dec. 21, 2001

(51) Int. Cl. C12Q 1/70; G01N 33/53; G01N 33/554; G01N 33/569

(52) U.S. Cl. 436/548; 435/5; 435/7.1; 435/975; 435/7.32

(57) ABSTRACT

Diagnostic methods and devices are provided to aid health-care professionals and non-professionals to determine whether a person's upper respiratory ailments are caused by a viral infection, bacterial infection, fungal infection, and/or allergy. In one embodiment, the method comprises contacting a sample to a surface that is printed with a binder that will bind, react or otherwise associate with a particular biomarker for these causes (e.g., bacterial infection) and diffract light that is reflected off of or that is transmitted through the printed surface. In another embodiment, the method comprises contacting a sample to a surface that is printed with a binder that will bind, react or otherwise associate with IgE antibodies to diffract light that is reflected off of or that is transmitted through the printed surface. In yet another embodiment, the method comprises contacting a sample to a surface that is printed with a binder that will bind, react or otherwise associate with a biomarker indicative of a viral infection (e.g., anti-Influenza A antibodies) and diffract light that is reflected off of or that is transmitted

### CROSS-REFERENCES TO RELATED APPLICATIONS

This application claims the priority of (1) U.S. patent application entitled “Diagnostic Device, System and Method” and further identified as Express Mail Label no. EL188516562US, filed Dec. 21, 2001, and (2) U.S. patent application entitled “Sensors and Methods of Detection for Proteinase Enzymes” and further identified as Express Mail Label no. EL602999586US, filed Dec. 21, 2001. The complete text, claims and drawings of all of the above applications are incorporated herein by reference in their entirety.

### FIELD OF THE INVENTION

The present invention relates to methods and devices that can be used to detect for the presence of a specific analyte or a specific class of analytes in a sample. Particularly, the present invention relates to methods and devices for detecting one or more analytes that will help users of the methods and devices determine whether an ailment is related to an allergy, a bacterial infection, a viral infection or a fungal infection.

### BACKGROUND

Persons suffering from upper respiratory symptoms, such as sneezing, coughing, congestion, runny nose, etc. often have difficulty determining the cause or causes of their symptoms. Any and all of the above-mentioned afflictions may be symptoms of one of a variety of illnesses. Any one of the following may cause these upper respiratory symptoms: a viral infection, such as cold or influenza; bacterial infection, such as pneumonia; an allergy; or a fungal infection, such as Aspergillus. Although these illnesses produce similar symptoms, the illnesses are very dissimilar and are treated differently.

An example of an upper-respiratory bacterial infection is sinusitis, which may be caused by any of a variety of bacteria, such as Mycoplasma pneumonia, Streptococcus, Haemophilus, Cataralus, etc. Generally, bacterial infections can be treated with antibiotics. Thus, persons suffering from a bacterial infection should visit a physician so appropriate antibiotics can be selected and prescribed. However, antibiotics usually cannot cure viral infections, such as cold and flu. The viral infections are usually allowed to run their course, and their symptoms will eventually subside. Examples of viruses that can cause upper respiratory viral infections include Influenza A, Influenza B, respiratory synctial virus (RSV), Rhinovirus, pare influenza, adenovirus, etc. The typical procedure for treating colds and flus is to comfort the patient by treating the symptoms rather than the disease. Treatments for the symptoms of cold and flu include the use of pain relievers, such as aspirin, acetaminophen and ibuprofen, to relieve aches and fever; decongestants and antihistamines to relieve congestion; and rehydrating by drinking liquids. Allergies are caused by sensitivity to one or more allergens. Common allergens include dog and cat dander, dust mites, pollens, molds, and some food components, such as milk, wheat, egg, soy, and shellfish. Allergies may be treated with the use of antihistamines or other long-term treatments. Fungal infections are caused by one or more fungi that can invade the upper respiratory tract and/or sinuses. A common example is Aspergillus. These infections are typically treated with an anti-fungal, such as azole compounds (e.g., miconadazole).

Unfortunately, no convenient and easy-to-use method or device exists for helping professionals or non-professionals determine whether a person is suffering from a bacterial infection, a viral infection, a fungal infection, and/or an allergy. What is needed is a simple, easy to use method or a device that will aid healthcare professionals and non-professionals alike to differentiate between these illnesses and to help determine if a person is suffering from a bacterial infection, a viral infection, a fungal infection, or an allergy.

### SUMMARY OF THE INVENTION

The present invention provides a method for determining the cause of a malady in animal in a sample comprising the steps of: providing a test device comprising obtaining a sample from the animal and exposing the sample to a plurality of discrete test sites that are provided on an integral test device. At least one of the test sites comprises a binder that is adapted to bind to at least one species or analyte selected from any of groups (i)-(v) wherein group (i) comprises a bacteria and substances produced by an animal in response to a bacterial infection, group (ii) comprises viruses and substances produced by an animal in response to a viral infection, group (iii) comprises fungi and substances produce by an animal in response to a fungal infection and group (iv) comprises protozoa and substances produced by a body in response to a protozoan infection and group (v) comprises substances produced by an animal in response to an allergic reaction; introducing a sample to the device contacting at least a portion of the sample to the test sites; and evaluating the test sites for a change indicating that binding has occurred and the analyte is present in the sample. For example, one of the binders such as he first binder can be selected so that the binder binds bacteria in general, a bacterium, a class or bacteria or a substance that is produced by an animal in response to a bacterial infection. Another binder at another test site may be select to bind to another bacterium, another class of bacteria or another substance that is produced by an animal in response to a bacterial infection or bind to one or more specie, class, or substance from one of the other groups (i) to (iv).

The present invention also provides a device for detecting the presence of a microorganism or an allergen in a sample wherein the device comprises a surface comprising at least two discrete test sites that comprise a binder that is adapted to bind to at least one analyte selected from any of groups (i)-(v), wherein group (i) consists of bacteria and substances produced by an animal in response to a bacterial infection, group (ii) consists of viruses and substances produced by an animal in response to a viral infection, group (iii) consists of fungi and substances produced by an animal in response to a fungal infection and group (iv) consists or protozoa and substances produced by an animal in response to a protozoan infection and group (v) consists of substances produced by an animal in response to an allergic reaction.

In one particularly advantageous embodiment, the methods of the present invention provide a diffraction-based diagnostic method for differentiating the cause of an respiratory infection by determining the presence of a microorganism or an allergen in a sample comprising: providing a test device comprising at least two test sites comprises a binder that is adapted to bind to at least one first analyte selected from any of groups (i)-(v) wherein the binders are different; introducing a sample to the device wherein at least a portion of the sample contacts the test sites; directing light at the test sites; and determining if light is diffracted from each of the test sites.

Features, aspects and advantages of the present invention will become better understood with reference to the following description and the appended claims. The accompanying drawings, which are incorporated in and constitute a part of this specification, illustrate several examples of the invention and, together with the description, serve to explain the principles of this invention.

### BRIEF DESCRIPTION OF THE FIGURES

The invention is hereinafter more particularly described by way of examples with reference to the following drawings in which:

FIGS. 1 and 1A are perspective views of a first diagnostic device and a second illustrated diagnostic device, respectively.

FIGS. 2 and 2A are exploded, perspective views of the first and the second diagnostic devices.

FIGS. 3 and 3A are plan views of the first and the second diagnostic devices.

FIGS. 4 and 4A are cross-sectional views of the first and the second diagnostic devices taken along line 4 of FIGS. 3 and 3A, respectively.

FIG. 5 is a perspective view of a third illustrated diagnostic device.

FIG. 6 is an exploded, perspective view of the third diagnostic device.

FIG. 7 is a plan view of the third diagnostic device.

FIG. 8 is a cross-sectional view of the third diagnostic device taken along line 8 of FIG. 7.

FIG. 9 is a perspective view of a fourth illustrated diagnostic device.

FIG. 10 is an exploded, perspective view of the fourth diagnostic device.

FIG. 11 is a plan view of the fourth diagnostic device.

FIG. 12 is a cross-sectional view of a fourth diagnostic device taken along line 12 of FIG. 11.

FIG. 12A is cross-sectional view of an alternate device that is a modification of the device illustrated in FIGS. 9-12.

FIG. 13 is a perspective view of a fifth illustrated diagnostic device.

FIG. 14 is an exploded, perspective view of the fifth diagnostic device.

FIG. 15 is a plan view of the fifth diagnostic device.

FIG. 16 is a cross-sectional view of the fifth diagnostic device taken along line 16 of FIG. 15.

FIG. 17 is a perspective view of a sixth illustrated diagnostic device.

FIG. 18 is an exploded, perspective view of the sixth diagnostic device.

FIG. 19 is a cross-sectional view of sixth diagnostic device taken along line 19 of FIG. 20.

FIG. 20 is a plan view of the sixth diagnostic device.

FIG. 21 is a cross-sectional view of a seventh illustrated diagnostic device.

Repeated use of reference characters in the present application and drawings is intended to represent the same, similar or analogous features or elements of the invention.

### DETAILED DESCRIPTION

Although the present invention is described in the context of several specific examples, configurations and embodiments, it will be appreciated that further combinations or alterations of the examples and methods described herein may be made by one skilled in the art without departing from the spirit and scope of the present invention. In addition, although reference is often made with respect to diagnostic methods and devices for detecting one particular biomarker for bacterial infections, those skilled in the art will appreciate that other modifications may be made to adapt the methods and devices for use with other analytes or biomarkers and for infections other than bacterial infections and for symptoms other than upper respiratory ailments. In the following discussion, reference is made to several figures to illustrate a few specific examples and embodiments of the present invention, which is provided by way of explanation of the invention and is not meant as a limitation of the invention.

In one embodiment, the present invention provides diagnostic methods and devices that may be used to detect an allergic condition in general or to determine the specific allergens causing the allergic condition. In another embodiment, the present invention provides diagnostic methods and devices that may be used to detect the presence of a bacterial infection in general or an infection related to a particular bacterium or a class of bacteria. In yet another embodiment, the present invention provides diagnostic methods and devices that may be used to detect the presence of viruses in general, a particular virus or a class of viruses in a sample.

In one particular embodiment, the present invention provides diagnostic methods and devices that may be used to detect an allergic reaction by detecting the presence of IgE antibodies in a sample. Advantageously, methods and devices of the present invention may be used by health-care professionals and by non-professionals to differentiate between illnesses and to determine the cause or causes of upper respiratory ailments. The sample may include one of more of the following or a component of one or more of the following: blood, serum, plasma, interstitial fluid, sweat, mucous, nasal secretions, saliva, vomitus, tears, lachrymal fluid, urine, fecal matter, vaginal discharge, vaginal secretion, seminal fluid, menses, sputa, fluid from a lung, cerebrospinal fluid, other body fluids, fluids from cells, organs or tissues and so forth. It is particularly desirable that the sample is fluid or dissolved or suspended in a fluid.

As used herein, a “fluid” includes a liquid, a gas, a solution, mixtures of gases and/or liquids, solutions, emulsions and/or suspensions and may comprise undissolved particles or other solids and may further include homogeneous or heterogeneous mixtures comprising at least one fluid. A sample that is not fluid, for example fecal matter, may be dissolved, or at least partially dissolved, in a fluid, for example distilled water or buffer, to provide a sample that is liquid so that the sample can be readily contacted to the binder-coated surface for testing. As used herein, “binding energy” is defined as the net energy required to decompose the bond between a binder and an analyte of a binder/analyte complex or otherwise dissociate the binder/analyte complex. It is desirable that the binder and the analyte that the binder binds have a binding affinity that is at least, 1×10⁴ M⁻¹ (liter per mole), and more desirably a binding affinity that is at least 1×10⁷ M⁻¹.

Methods of detecting analytes and systems and devices that detect analytes via the formation of a diffraction image are disclosed and described in the U.S. patents and International PCT applications discussed herein. Methods, systems and devices that detect the presence of an analyte by detecting the formation of a diffraction image provide a simple method for determining the presence of an analyte.

Examples of methods, systems and devices for detecting an analyte via the formation of a diffraction image are disclosed and described in U.S. Pat. No. 5,922,550, U.S. Pat. No. 6,020,047, U.S. Pat. No. 6,221,579 and International Publication No. WO 98/27417 which are hereby incorporated by reference herein in their entirety. The devices described in the above-referenced documents can be produced by printing a species onto a surface. The species is selected to bind, react or otherwise associate with an analyte of interest and is referred to herein as a “binder”. A binder may include any chemical species, compound, composition, moiety, particle and so forth that will bind, react or otherwise associate with the analyte of interest. Preferably, the binder is specific to the analyte of interest or a class of analytes of interest and does not appreciably bind, react or otherwise associate with any other matter that may be found in the sample of interest. The binder can be any analyte-specific receptor material that can be printed onto a substrate and that will specifically bind to an analyte of interest.

Thus, the binder is one part of a specific binding pair with the analyte; examples of analyte/binder pairs include, but are not limited to: antigen/antibody, such as IgE antibody/anti-IgE antibody; antibody/antibody-binding protein (e.g., Protein A or Protein G); enzyme/substrate; oligonucleotide/DNA; chelator/metal; enzyme/inhibitor; bacteria/receptor; bacteria/antibody to bacterial cell markers; or bacteria/anti-CRP antibody; virus/receptor or Influenza A and anti-Influenza A antibodies; fungus/anti-Aspergillus antibody; cellular toxin/receptor; cellular toxin/antibody to toxin; fungus/receptor; hormone/receptor; DNA/RNA, or RNA/RNA; oligonucleotide/RNA; and binding of these species to any other species, as well as the interaction of these species with inorganic species. The binder material that is printed onto the substrate is characterized by an ability to specifically bind the analyte or analytes of interest. The variety of materials that can be used as a binder material are limited only by the types of material which will combine selectively (with respect to any chosen sample) with the analyte. Subclasses of materials which can be included in the overall class of receptor materials includes toxins, antibodies, antigens, hormone receptors, parasites, cells, haptens, metabolites, allergens, nucleic acids, nuclear materials, autoantibodies, blood proteins, cellular debris, enzymes, tissue proteins, enzyme substrates, coenzymes, neuron transmitters, viruses, viral particles, microorganisms, proteins, saccharides, chelators, drugs, and any other member of a specific binding pair. This list only incorporates some of the many different materials that can be printed onto the substrate to produce a diagnostic device. Whatever the selected analyte of interest is, the binder is designed to bind, react or otherwise associate with the analyte(s) of interest.

Generally, the binder is printed onto a substrate, for example a plastic film, in a defined pattern such that the binder-printed film does not diffract electromagnetic radiation when the electromagnetic radiation is reflected off of or transmitted through the binder-printed film but diffracts electromagnetic radiation after the binder-printed film is exposed to the analyte and the analyte has bound, reacted or otherwise associated with the binder. Alternatively, the binder-printed film or surface may exhibit a measurable increase or decrease in diffraction after exposure to the analyte. For example, a film may be printed with a binder such that the binder-printed film does not diffract light but does diffract after an analyte binds, associates or otherwise reacts with the binder-printed surface. In another example, the binder-printed film initially diffracts light but does not diffract light or diffracts less after an analyte binds, associates or otherwise reacts with the binder-printed surface. In yet another example, the film may be printed with a binder so that binder-printed film initially diffracts light but when the analyte binds with binder-printed surface, light is diffracted to a measurably greater extent. Thus, the presence of analyte can be determined by a measurable change in diffraction of light that is transmitted through or reflected off of the substrate surface. If light or other electromagnetic radiation is to be transmitted through the surface of a film to detect diffraction, it is desirable that the film is transparent or at least partially transparent to the light or other electromagnetic radiation that will be used to detect diffraction.

Devices of the present invention include a surface or at least a portion of a surface that is printed with a binder. The printing of the surface may be accomplished by microcontact printing the binder onto the surface in a defined pattern. Microcontact printing is desirable and allows printing of patterns with size features of about 100 μm and smaller. Features in this size range are desirable for diffraction when the electromagnetic radiation wavelength is in the spectrum of visible light, from about 4000 Angstroms to 7000 Angstroms. However, it is noted that light over other wavelengths, both longer and shorter wavelength electromagnetic radiation, may be used to detect diffraction. A pattern of binder allows for the controlled attachment of analyte or analyte receptor. An elastomeric stamp may be used to transfer binder to a surface. If the stamp is patterned, a patterned binder layer will be printed on the surface when the stamp is wet with the binder, dried, and then contacted with the surface.

Gold-coated, printed films that produce diffraction patterns and methods of contact printing such films are described and disclosed in U.S. Pat. No. 6,020,047 and U.S. Pat. No. 6,048,623, which are hereby incorporated by reference herein in their entirety. U.S. Pat. Nos. 6,020,047 and 6,048,623 describe methods of microcontact printing self-assembling monolayers that allow for the selective placement of reagents that can react chemically or physically with an analyte or a group of analytes that are of interest to produce a diffraction image.

Generally, an analyte may be any stimulus including but not limited to any chemical or biological species, compound, composition, moiety, particle, and so forth that that will bind, react or otherwise associate with the binder or with which the binder will respond. Analytes that are contemplated as being detected include, but are not limited to, one or more the following: species of bacteria, including, but not limited to, Hemophilis, Neisseria meningitides serogroups A, B, C, Yand W135, Streptococcus pneumoniae; yeasts; fungi; viruses including, but not limited to, Haemophilus influenza type B or RSV; rheumatoid factors; antibodies including, but not limited to, IgG, IgM, IgA and IgE antibodies; antigens including, but not limited to, streptococcus Group A antigen, streptococcus Group B antigen, viral antigens, fungal antigens, an antigen derived from microorganisms, antigens associated with autoimmune diseases, influenza and tumors; allergens; enzymes; hormones; saccharides; proteins, such as C-reactive protein (CRP); lipids; carbohydrates; drugs including, but not limited to, drugs of abuse and therapeutic drugs, nucleic acids; haptens, environmental agents, other blood-born disease markers; substances produced by an animal in response to a bacterial, viral, fungal or protozoan infection; and so forth.

A binder may be microprinted on a polymer film or other substrate. Desirably, a binder is selected and printed that is an analyte-specific receptor material and specifically binds to the analyte or class of analytes of interest. Thus, the binder material and analyte are defined as a specific binding pair with the analyte; examples of analyte/binder pairs include, but are not limited to, antigen/antibody, antibody/antibody-binding protein, enzyme/substrate, oligonucleotide/DNA, chelator/metal, enzyme/inhibitor, bacteria/receptor, virus/receptor, cellular toxin/receptor, fungus/receptor, hormone/receptor, DNA/RNA, or RNA/RNA, oligonucleotide/RNA, and binding of these species to any other species, as well as the interaction of these species with inorganic species. The binder material that is printed on to a substrate layer is characterized by an ability to specifically bind the analyte or analytes of interest. The variety of materials that can be used as a binder material are limited only by the types of material which will combine selectively (with respect to any chosen sample) with the analyte. Subclasses of materials which can be included in the overall class of binder materials include toxins, antibodies, antigens, hormone receptors, parasites, cells, haptens, metabolites, allergens, nucleic acids, nuclear materials, autoantibodies, blood proteins, cellular debris, enzymes, tissue proteins, enzyme substrates, coenzymes, neuron transmitters, viruses, viral particles, microorganisms, proteins, saccharides, chelators, drugs, and any other member of a specific binding pair.

A widely used biomarker that indicates a bacterial infection is C-reactive protein (CRP). A concentration of less than or equal to 10 micrograms per milliliter of serum is considered normal. Levels or concentrations of CRP higher than about 10 μg/mL can be an indication of a bacterial infection. Levels of CRP above 40 μg/mL are highly predictive of a bacterial infection in the host. Levels above 50 μg/mL are even more predictive of a bacterial infection. CRP may be found in blood, sera, menses, vaginal fluid, nasal secretions, cerebrospinal fluid, saliva, sputa, tears, sweat, urine, and so forth; it should be noted that the normal range may vary for each body fluid. Another suggested biomarker that indicates a bacterial infection is neutrophil lipocalin. The human body produces these substances in response to bacterial infections; therefore, bacterial infections can be identified by increased concentrations of these substances.

To determine if a person is suffering from a bacterial infection, a sample of the person's blood, serum, nasal secretion or other body fluid may be tested for CRP or another biomarker that indicates bacterial infection. Other potential biomarkers for bacterial infections include, but are not limited to: immunocalins such as human neutrophil lipocalin (HNL); cytokines and associated materials, such as IL-6 or IL-2R (soluble receptor of IL-2); enzymes, such as plasma neutrophil elastase; acute phase proteins, including CRP; and other proteins, such as procalcitonin; and so forth. Additionally, antibodies to bacteria, a class of bacteria or a specific bacterium may be used as a binder to detect bacteria, a class of bacteria or the specific bacterium, respectively. For example, a test that is specific to Strep A (Streptococcus group A) can be developed if anti-Strep A antibodies are identified or developed and used as a binder. Another option would be to use an antibody or binder for enzymes such as ESBL, or extended spectrum beta-lactamase, which can be indicative of antibiotic-resistant strains of bacteria. An antibody to CRP may be used as a binder and printed in a pattern on to a substrate to detect the presence of CRP in a sample and thus a bacterial infection in general. Specific antibodies, such as those for CRP, can be purchased from a variety of suppliers. An exemplary listing of such suppliers is published in Linscott's Directory.

A biomarker that can be used reliably to indicate an allergic condition is IgE. IgE is an immunoglobin that can be found in the blood stream or serum and is typically at levels below 350 ng/mL. A person's IgE levels rise when that person is suffering from an allergic reaction or condition. If IgE concentrations in blood are found at elevated levels, for example 350 ng/mL or greater, this is an indication of an allergic condition. Anti-IgE antibody may be used as binder for IgE. An anti-IgE antibody that is F_{c} specific, that is an antibody that binds to the F_{c} region of IgE, may be desirable as one of the binders for IgE and thus a marker for allergic reactions or conditions. It may be desirable to use an allergen or an allergen extract as binder to indicate allergies to a particular allergen. For instance, mold mix extract may be used as a binder to determine if a person is suffering from an allergic reaction to mold mix. The mold mix extract as a binder would only capture IgE that is specific to that allergen (e.g., mold mix specific IgE); subsequent binding by particles labeled with anti-IgE (e.g., antibodies to Fc region of IgE) would complete the assay.

Three systems to test for allergies could be used; the first two would be for an allergen-specific test to determine what allergen(s) a person is allergic: one in which the capture binder is allergen with anti-IgE coated particles, and the other in which the capture binder is anti-IgE with allergen-coated particles. An alternate system could be used to test for total IgE as more of a yes/no test for allergies, which would use anti-IgE as both the capture binder and the antibody on the particles. Alternatively, a mixture of common allergens could be used as one of the binders to conduct a yes/no test for allergies. Two possible systems are as follows: one possible system would have the allergen mixture as capture binder with anti-IgE coated particles, and another system could use anti-IgE as the capture binder with allergen mix-coated particles.

Biomarkers for allergic conditions include, but are not limited to: cytokines, such as IL-4; eosinophil-based proteins, such as eosinophilic cationic protein (ECP), eosinophil neutrotoxin or major basic protein; histamine; leukotrienes; lysozyme; myeloperoxidase; elastase, tryptase; or endothelin; and so forth. Additionally, a mixture of allergens can be employed as a binder to detect an allergic reaction to the allergen or allergen(s) contained in the mixture. The mixture of allergens could include one or more of the following allergens: pollens, such as tree, grass and ragweed pollen; molds, cat and dog dander; dust mites; food products, such as egg and milk; and so forth. Sources of allergens include ALK-Abello, Inc. of Wallingford, Conn. and the National Institute for Biological Standards and Controls of the United Kingdom.

A listing of suppliers and a listing of various antibodies that are commercially available are provided in Linscott's Directory (1998) pp. 1-207. Examples of pairings of specific binders and specific analytes or specific classes of analytes that can be detected via the use of a specific binder are known and are known to persons skilled in the art and can also be obtained from various sources including Linscott's Directory which is hereby incorporated by reference.

Viral infections may be detected by negative results for both bacterial infections and allergic condition. For example, if a person is suffering symptoms that indicate a possible allergic condition or a viral or bacterial infection, and then tests negative for both bacterial infections and allergic conditions, that person likely is suffering from a viral infection. However, it may be desirable to test for specific viral infections. Such a test can be developed if a binder for the antibodies produced due to that particular viral infection is identified or produced and used as a binder in accordance with the descriptions contained herein. For example, antibodies to anti-Influenza A antibodies may be identified and used as a binder in the devices described and references incorporated herein to provide a device and a test or method for identifying infection by Influenza A virus.

The methods and devices discussed herein provide a test for detecting a bacterial infection and may be used to determine the cause of, for example, upper respiratory problems. Such devices and methods have particular use in health1E care applications. Analytes may be detected in a variety of sample media including, but not limited to, blood, urine, menses, vaginal secretions, nasal secretions, saliva and so forth. If a sample media is not fluid, it may be desirable to dissolve at least a portion of the sample in a fluid.

U.S. Pat. No. 6,180,288 and International Publication No. WO 98/43086 disclose and describe the use of one or more responsive gels coated on a patterned self-assembling monolayer and the use of such devices. The responsive gels described therein react or respond to a stimulus, i.e. an analyte, to produce a diffraction image. U.S. Pat. No. 6,180,288 and International Publication No. WO 98/43086 are both hereby incorporated by reference herein in their entirety.

Diffraction-based detectors and methods of detection using optical diffraction that do not require self-assembled monolayers are disclosed and described in U.S. Pat. No. 6,060,256 and International Publication No. WO 99/31486. U.S. Pat. No. 6,060,256 and International Publication No. WO 99/31486 are hereby incorporated by reference herein in their entirety. U.S. Pat. No. 6,060,256 and International Publication No. WO 99/31486 also disclose and describe the optional addition of nutrients for a specific class of microorganisms with such diagnostic devices, systems and methods to provide for the detection of lower concentrations of analytes.

U.S. Pat. No. 6,221,579 and International Publication No. WO 00/34781 disclose and describe the addition of diffraction enhancing elements. Diffraction enhancing element particles that may be used with the present invention include, but are not limited to, glass, cellulose, synthetic polymers or plastics, latex, polystyrene, polycarbonate, bacterial or fungal cells, metallic sols, and so forth. A desirable particle size ranges from a diameter of approximately 0.04 μm to 100.0 μm. The composition of the element particle and structural and spatial configuration of the particle is not critical to the present invention. However, it is desirable that the difference in refractive index between the medium and the enhancing element is between 0.1 and 1.0. Diffraction enhancing elements are optionally included in such devices, systems and methods to provide for the detection of smaller species of analyte, such as proteins, DNA, RNA, other low molecular weight analytes and low molecular weight surface markers on organisms. U.S. Pat. No. 6,221,579 and International Publication No. WO 00/34781 describe the modification of microspheres so that the microspheres are capable of binding with a target analyte and to the device surface. The microspheres are capable of producing a substantial change in height and/or refractive index to enhance diffraction, thereby increasing the efficiency of such devices, systems and methods and can provide for the detection of smaller species of analyte. U.S. Pat. No. 6,221,579 and International Publication No. WO 00/34781 are hereby incorporated by reference herein in their entirety.

International Publication No. WO 00/36416 describes and discloses devices and systems comprising a patterned deposition of antibody-binding proteins for detecting antibodies. International Publication No. WO 00/36416 is also hereby incorporated by reference herein in its entirety.

There is a need to provide diagnostic methods and devices that are easy to use by health-care professionals and nonprofessionals, including consumers, to determine if an infection is bacterial or viral or if the symptoms are related to an allergy. Methods and devices and systems of the present invention provide a method of determining if a mammal, particularly a human, is suffering from a bacterial or a viral infection or an allergy. Advantageously, these methods and devices may be used by individuals at home to monitor health-related conditions. It is also desirable to provide a device that provides for detection of bacterial infections and allergic reactions. Such methods and devices are provided in at least one embodiment of the present invention. In yet other embodiments, the present invention provides a device that is portable and compact and has no single dimension greater than 20 inches, more desirably no greater than 10, and most desirably no greater than 6 inches. Advantageously, devices of the present invention can be configured so that they are portable and hand-held and the methods can be performed at home.

By way of example, a device that detects bacterial infections can be provided by printing a CRP antibody onto a test surface in the manners disclosed and described in the previously incorporated patents and international publications which are commonly owned and assigned to the assignee of the present invention. A guide or other means for directing a sample to a test surface may be used to facilitate the transport of a blood sample from a freshly lanced finger or other body site to a CRP antibody-printed diagnostic test surface. Examples of guides and means for directing a sample to a test surface include, but are not limited to the following: capillaries, conduits, tubular structures, channels, slots, parallel plates, grooves and other types of openings, passages or penetrations, porous materials of various shapes and configurations, surfaces having varying degrees of surface energy or hydrophobicity, pumps, vacuums, suction, air pressure, electrostatic attraction or repulsion, hydrophobic or hydrophilic interaction, electromagnetic coercion, osmotic pressure, centripetal acceleration, localized heating or cooling, charged gas bladders and so forth. The cross-section of the guide or other means may be non-circular. Desirably, the guide or means for directing a sample from a source of the sample towards the surface of the substrate that is printed with a binder directs the sample toward the surface of the substrate that is printed with a binder through use of capillary forces or by capillary action. More desirably, the guide or means comprises a material and a structure that has an affinity for the sample that is greater than the affinity of the sample to the source from which the sample is obtained.

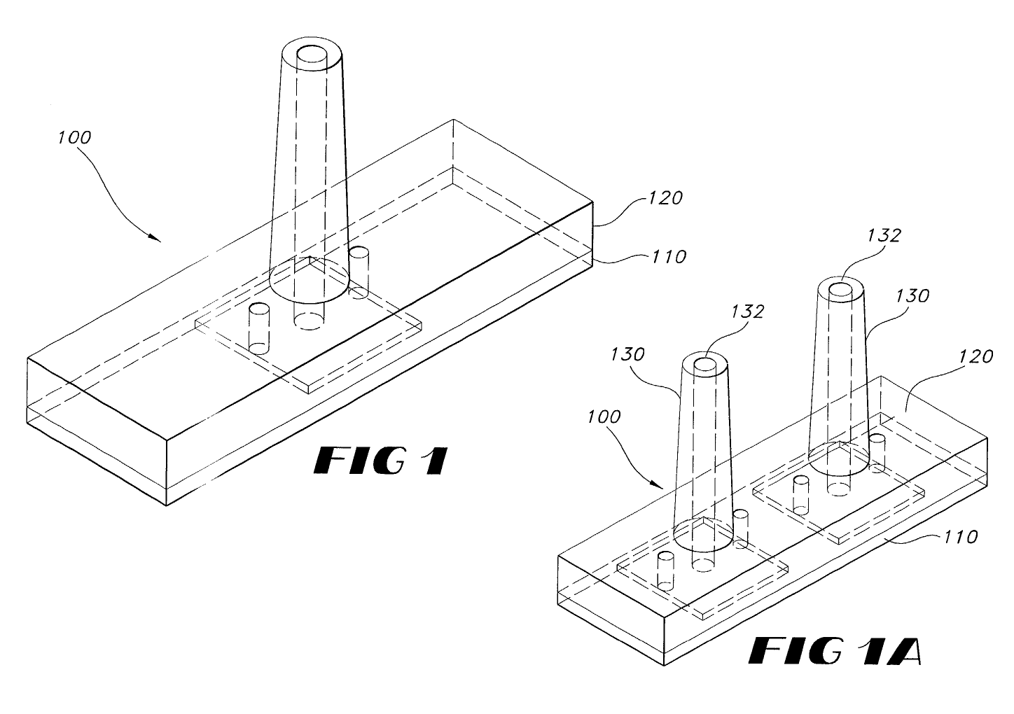

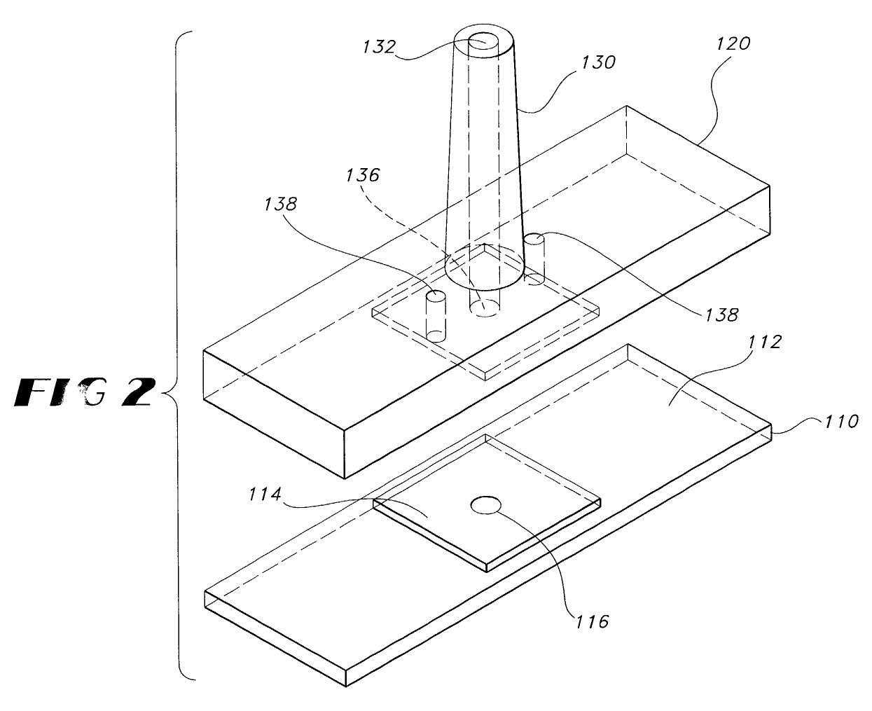

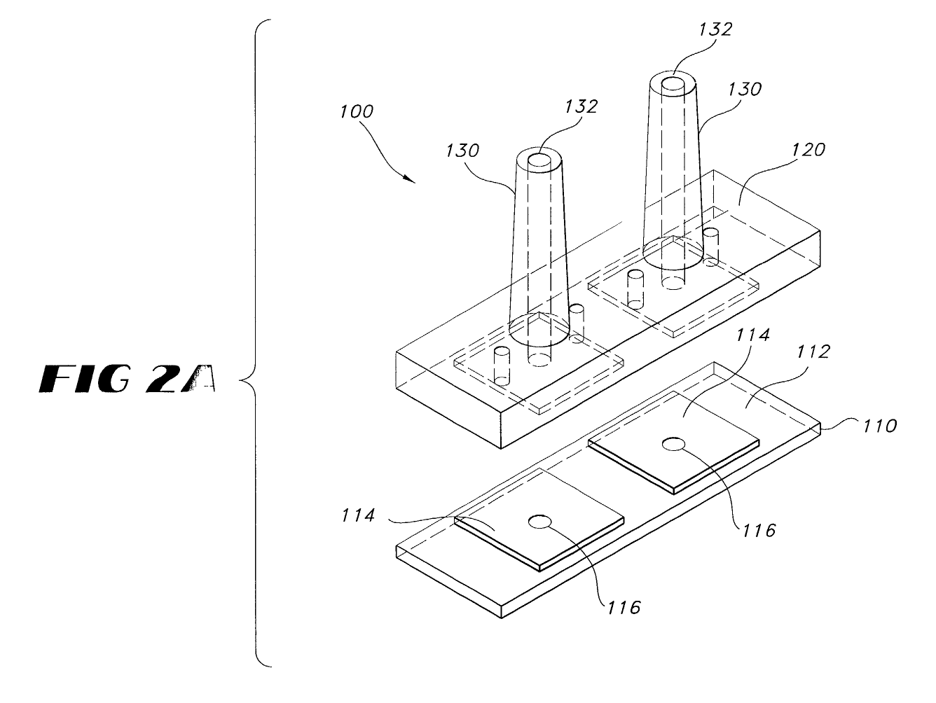

Examples that include one or more means for directing a sample to a test surface are illustrated in the accompanying figures and are described in detail herein by reference to the accompanying figures. In the examples illustrated in FIGS. 1-5, the diagnostic device may be a disposable test strip 100 that includes a film 110 that has a surface 112. At least a portion of the surface of the film is printed with a binder for an analyte (not shown). The binder-printed portion of the film is the portion of the film that is exposed to a sample to test for analyte in the sample. The portion of the surface that is printed with a binder may be printed by one of the methods described in the previously discussed patents and international publications that are commonly assigned and discussed above or by an ink-jet printing method. The use of ink-jet printing methods to manufacture diffraction-based diagnostic devices is described and disclosed in U.S. patent application Ser. No. 09/557,453 entitled “Use of Ink-Jet Printing to Produce Diffraction-K-C Based Biosensors” and filed on Apr. 24, 2000. U.S. patent application Ser. No. 09/557,453 entitled “Use of Ink-Jet Printing to Produce Diffraction-Based Biosensors” and filed on Apr. 24, 2000 is hereby incorporated by reference herein in its entirety.

The diagnostic device may further include a wicking agent. The wicking agent may be provided by a layer of wicking material over the binder-printed surface. Desirably, the layer of wicking agent is provided with an opening and the sample is deposited in or direct to the opening. The opening is also useful for transmitting light to the binder-printed surface. The diagnostic devices illustrated in FIGS. 1-4 and 1A-4A, further include an optional layer of wicking agent 114 to facilitate removal of a sample from the portion of the test surface that is printed with the binder after a desired incubation time. Examples of diagnostic devices and test strips that comprise a diffraction-based, diagnostic test locus are disclosed and described in the aforementioned patents and international publications which have incorporated by reference herein. Wicking agents and the use of wicking agents in conjunction with such diagnostic devices and methods are also disclosed and described International Publication No. WO 01/44813. The addition of a wicking agent or a layer of wicking agent in the devices of the present invention is suggested but optional and may improve contact of a sample that is to be tested for an analyte with the binder-printed surface of a diagnostic device, remove unbound diffraction-enhancing elements and/or remove excess sample from the binder-printed surface thus improving the reliability of diagnostic device and methods. Thus, a layer of wicking agent may be incorporated into a diagnostic device or test strip of the present invention to provide desired incubation time of a sample on a binder-printed surface, to remove unbound diffraction-enhancing elements or to eliminate the need to rinse or wash excess sample from the binder-printed surface before testing the surface with light or other electromagnetic radiation. International Publication No. WO 01/44813 is hereby incorporated by reference herein in its entirety. Examples of wicking agents include, but are not limited to polyolefins such as polypropylene, fluoropolymers such as polyvinylidene fluoride, nitrocellulose, cellulose, cellulose acetate, glass microfiber structures and so forth. The wicking agent may be provided as a layer over the binder-printed surface. The layer of wicking agent may be a nonwoven layer, a porous membranes, a semiporous membrane or so forth.

A device generally indicated as 100 may include a means 120 for directing a sample to a test surface. In the example illustrated in FIGS. 1-4, the means 120 for directing a sample to a test surface includes a capillary 130 that is used to direct a liquid sample that is placed near a first opening 132 through an interior passage 134 through the capillary 130 to a second opening 136 that is proximate to a layer of a wicking agent 114.

In an illustrative example, the device can be used to test blood for an analyte that may be contained in the blood, such as CRP, IgE or other analyte(s) of interest. An individual may test his or her blood for CRP by first pricking his or her finger (or other body site) and then contacting a blood droplet that is obtained to first opening 132. A portion of the blood droplet is then directed from the finger through interior passage 134 to the second opening 136 by capillary action. This brings blood into contact to the test surface 112 that has been printed with a CRP antibody. Another portion of the test surface may be printed with a binder for another biomarker for bacterial infection, such as IL-6, or a biomarker for allergic reactions, such as anti-IgE antibody. The layer of wicking agent 114 then draws the blood sample from second opening 136 and brings blood across the surface of the device that is printed with CRP antibody which provides a test for bacterial infection. The sample, or at least a portion of the sample, contacts the portion of the device that is printed with the antibody so that CRP contained in the sample is allowed to bind, react or otherwise associate with the antibody that is printed on the surface 112. If CRP is present in the blood sample, the CRP will bind with the binder and any optional diffraction enhancing elements and will diffract light. To detect for the presence of CRP, light is then transmitted through or reflected off of the surface to determine if the surface diffracts light. If the surface diffracts light, the blood sample contains CRP. The means 120 for directing a sample to a test surface may further comprise one or more means for venting pressure 138. Pressure may build up due to the movement of a sample through the means 120 for directing a sample to a test surface and may prevent further movement of the sample through the means 120.

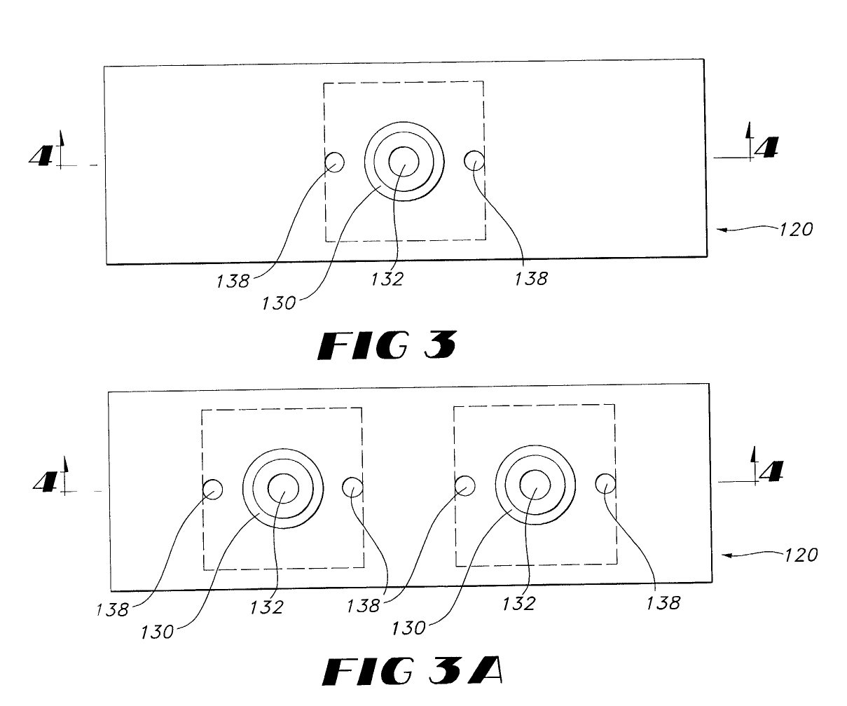

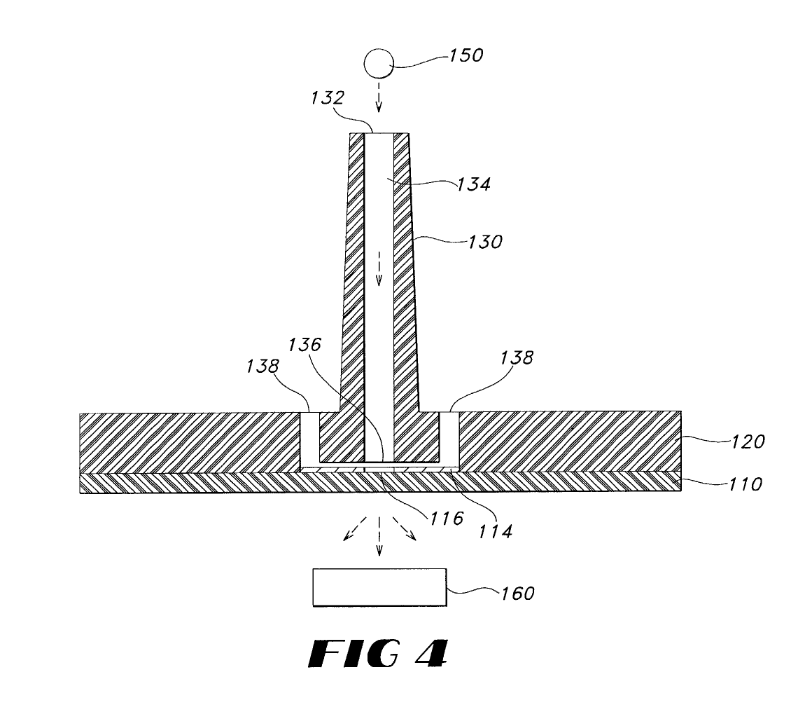

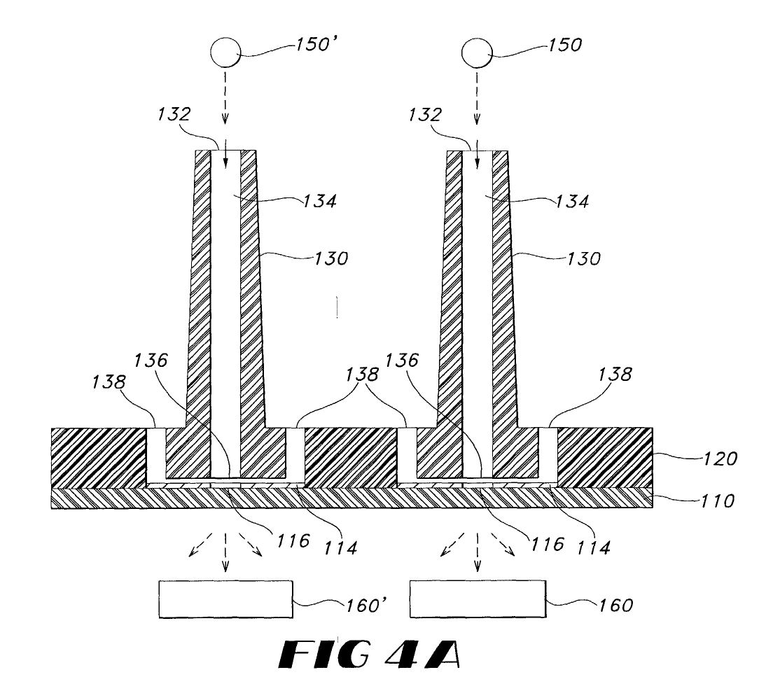

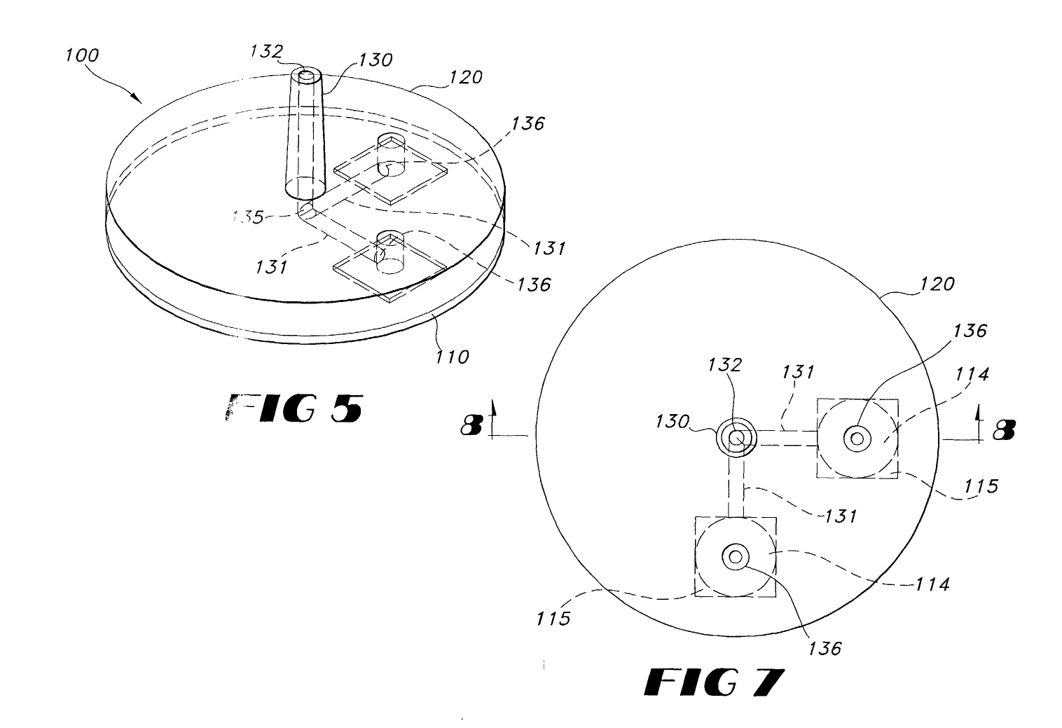

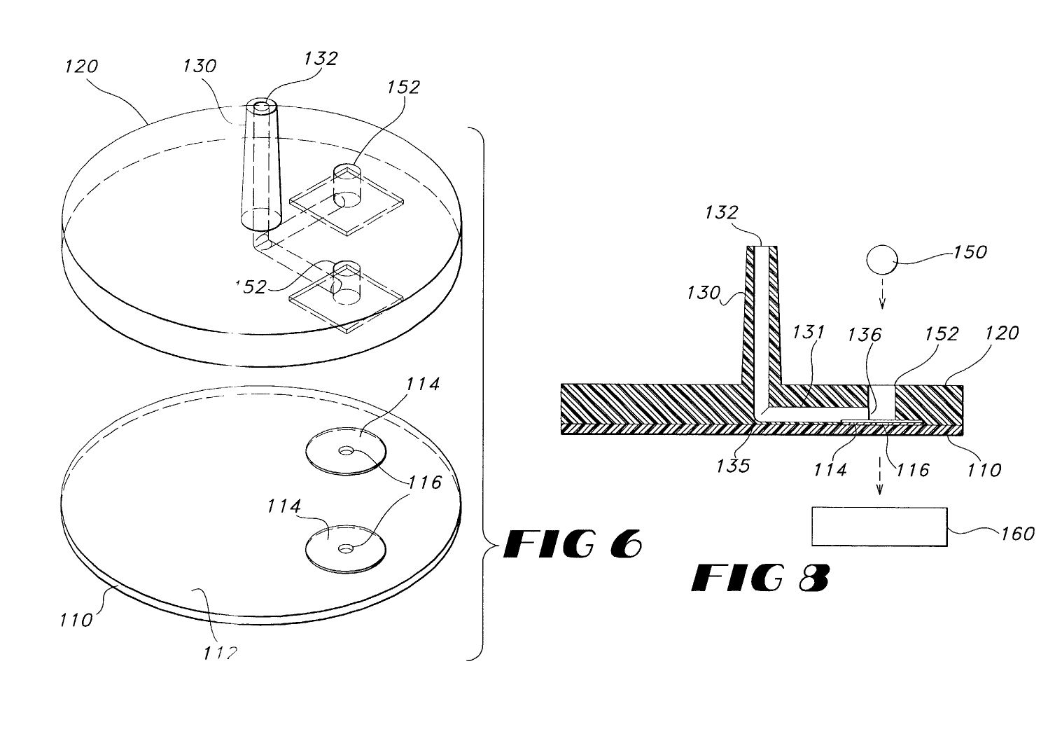

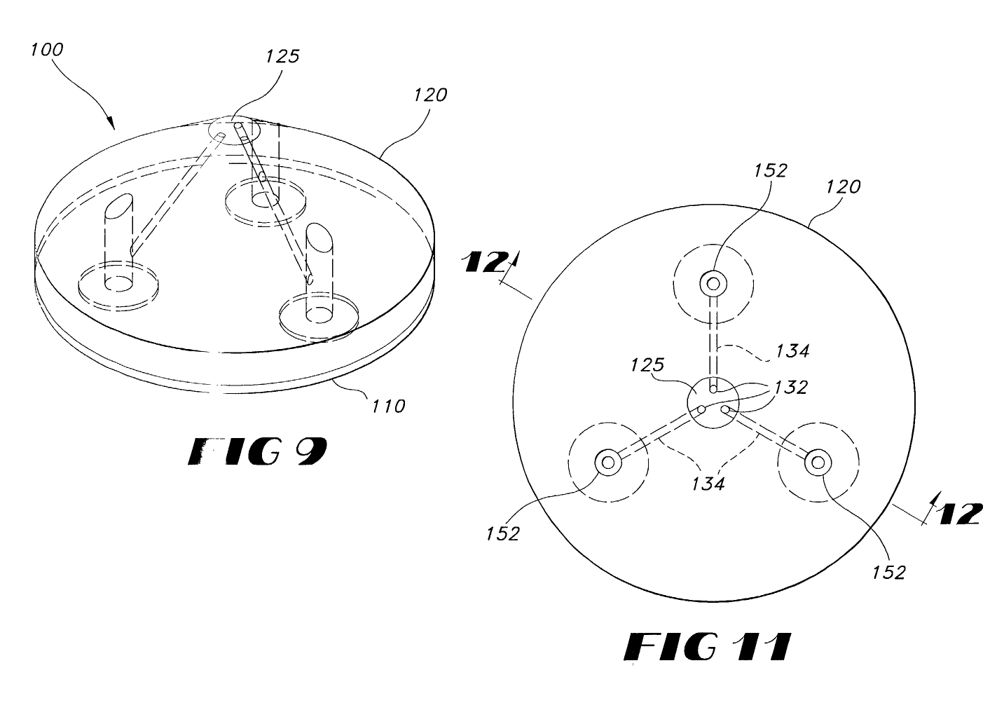

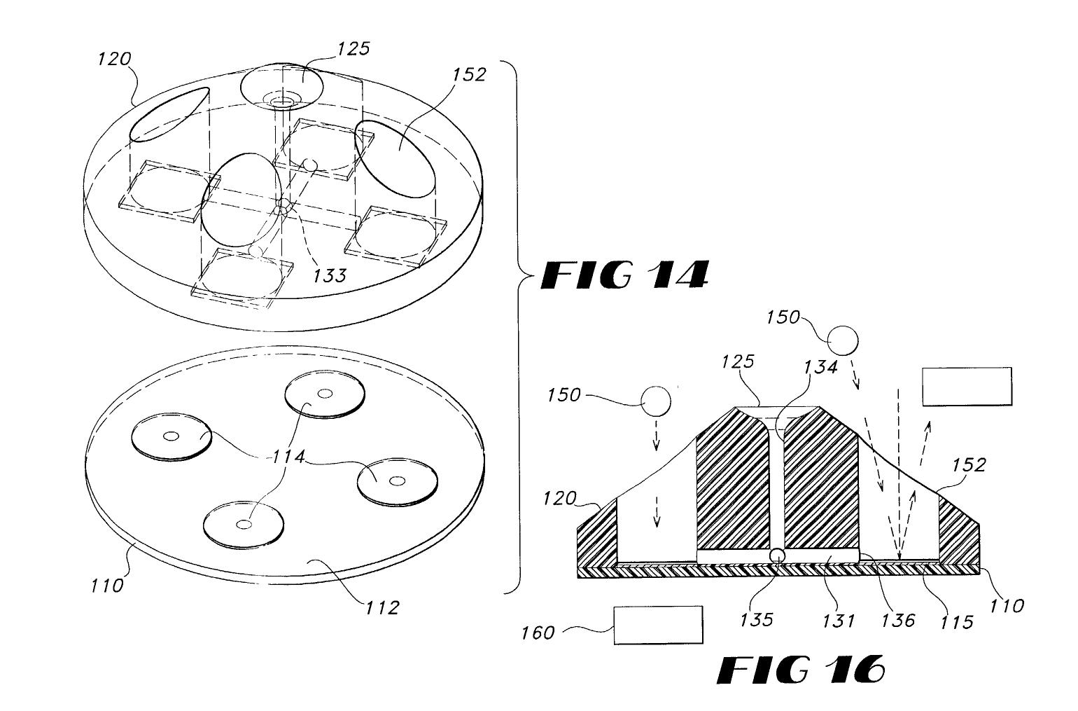

A system for detecting the presence of an analyte may be provided by including a light source 150 that can be directed through the interior 134 of the capillary 130 to the binder-printed surface or through another opening 152 or window that transmits light as illustrated in FIGS. 5-16. As illustrated in FIG. 4, light source 150 is configured to align with interior passage 134 so that light is transmitted through the test surface to produce a diffraction image. Light source 150 may be any source of light including ambient light. However, it is desirable that the light source is a focused light source such as a laser or point white light, or is focused via the use of a mechanical device such as a pin hole. It may also be further desirable that the light source or focused light source is a monochromatic light source, that is a light source that produces light of one wavelength. The wicking agent 114 may include an opening 116 through which light may be directed to the test surface. A system may also further include a detector for determining if diffraction occurs and, thus, analyte is detected. The detectors may be any device that measures light intensity or any device that can be used to determine between diffraction and non-diffraction. Examples of detectors or devices that may be used for detection include photodiodes, array detectors and other devices or means of measuring the intensity of the diffracted light. Diffraction may be detected by a human in embodiments that produce a visible diffraction pattern. Systems may also further include a housing for configuring and protecting various components of a system and to form a unified, consolidated system for detecting an analyte that is easy to use.

Detection of a target analyte in a sample may be determined by measuring a difference in diffraction of light from the binder-printed surface before and after the binder-printed surface is exposed to a sample. In most instances, the presence of analyte will be detected by determining if the test surface diffracts light or other electromagnetic radiation after the test surface is exposed to a sample. However, it is possible that the presence of analyte may be measured by either an increase or a decrease in diffraction or by lack of diffraction if a binder-printed surface is provided that initially diffracts electromagnetic radiation and will diffract electromagnetic radiation to a greater or a lesser extent, respectively, when analyte is bound, reacted or otherwise associated with binder that is printed on the surface.

Devices may be provided that direct sample to more than one test site. The multiple test loci may be provided on the same surface or film or on separate surfaces or films. An example of a device that directs a sample to two test loci is illustrated in FIGS. 1A-4A. The device illustrated in FIGS. 1A-4A comprises a means 120 for directing a sample to a test surface that comprises two capillaries 130 for directing sample to two test loci (not shown). Each capillary comprises a first opening 132, an interior passage 134, and a second opening 136 that is in fluid communication with a test site. A device that comprises two test loci may be used to test a sample for two different analytes, for example CRP and IgE, or to test a sample for the same analyte at two different test loci. In this case, one test location would have a test surface printed with a binder for CRP and coated with anti-CRP labeled particles, while the other test location would be printed with a binder for a biomarker of allergic reactions, such as anti-IgE antibody and coated with anti-IgE labeled particles. Thus, a device of the present invention may provide a back-up test or test a sample at one locus and the other, control, locus may be used as a base line for determining diffraction versus non-diffraction. For example, the second test locus can be used as a control locus and can be used to confirm that the device is functioning correctly. Alternatively, or in addition, the second test locus can act as a control test pattern by providing a benchmark diffraction pattern that must be achieved in order for a test result to be considered positive for the presence of analyte. A diagnostic test kit may include control samples that contain one or more samples of the target analyte(s). Thus, control sample may be used to confirm that the device functions properly. A kit may further comprise one or more solutions to assist in conducting the methods of the invention, for example, solutions for diluting samples, solutions for incubating samples, solutions for rinsing samples and solutions comprising one or more blocking agents. Desired solutions, control and otherwise, are sterile and free of substances that may interfere with detection of analyte.

In the examples illustrated in FIGS. 1-4 and 1A-4A, the diagnostic devices are illustrated showing a means 120 for directing a sample to the test surface comprises a capillary that is generally linear. However, a means 120 for directing a sample to the diagnostic test surface may be nonlinear. Means 120 for directing a sample to the diagnostic test surface that are curved or that comprise one or more turns or branches are illustrated in FIGS. 5-8, 13-16 and 21. The diagnostic devices illustrated in FIGS. 5-16 provide diagnostic devices having a means 120 for directing a sample to a test surface that direct a portion of a sample to more than one test location. These devices with multiple test loci can provide for the testing of one or more analytes by incorporating one or more binders specific to an analyte at the different test surfaces or at different portions or locations of a film surface. For example, a diagnostic device may be provided that divides a sample of blood and tests one portion of a sample at a first locus for one analyte, for example CRP and tests another portion of the sample at a second, different test locus for another analyte, for example IgE. Such a device provides a method of determining if a person is suffering from a bacterial infection or an allergy. And further, such a device provides an indication that a person with upper respiratory symptoms is suffering from a viral infection if both the bacterial test and the allergy test are negative. Additional analytes or classes of analytes, such as influenza, may be tested for at additional test loci.

Those skilled in the art will appreciate that other modifications may be made to adapt the diagnostic devices and methods of the present invention. A few modifications and adaptations are illustrated herein. FIGS. 5-8 illustrate a diagnostic device 100 in which the means 120 for directing a sample to a test surface divides a sample into two portions and directs the portions to two test loci that are located on the surface 112 of film 110 located under and in contact with wicking agent 114. The means 120 for directing a sample to a test surface illustrated in FIGS. 5-8 comprises one capillary 130 that connects to two diverging capillaries or channels 131 and 131 at intersection 135. Each capillary channel extends to an opening 136 or 136 that is proximate a test locus. The test loci are printed with a binder and may further include a wicking agent, more specifically a layer comprising a wicking agent 114. The layer of wicking agent may further comprise an opening 116 through which electromagnetic radiation may be directed to the test locus and binder printed surface to determine if diffraction occurs. A device of the present invention may include additional diverging channels and test loci.

Devices of the present invention may also include one or more openings 152 or windows that transmit light or other electromagnetic radiation over each test locus 115 so that light or electromagnetic radiation can be directed to and transmitted through or reflected from the binder-printed surface. The device illustrated in FIGS. 5-8 is used by bringing a liquid sample into contact with opening 132. The sample is then directed through interior passage 134 to intersection 135 where the sample diverges and a portion of the sample is directed to each of channels 131 and 131 and finally to the second openings 136 and 136. The layers of wicking agent 114 then draw a portion of the sample from each of the second openings 136 and spread the sample across the respective portion of the device that is printed with the binder(s). The sample, or at least a portion of the sample, then contacts the portion of the device that is printed with the binder antibody so that analyte that may be contained in the sample can bind, react or otherwise associate with the binder that is printed on the surface 112. Light or other electromagnetic radiation may then be directed through opening 152 to determine if the surface diffracts electromagnetic radiation either by reflecting electromagnetic radiation off of the surface 112 or transmitting electromagnetic radiation through the surface 112.

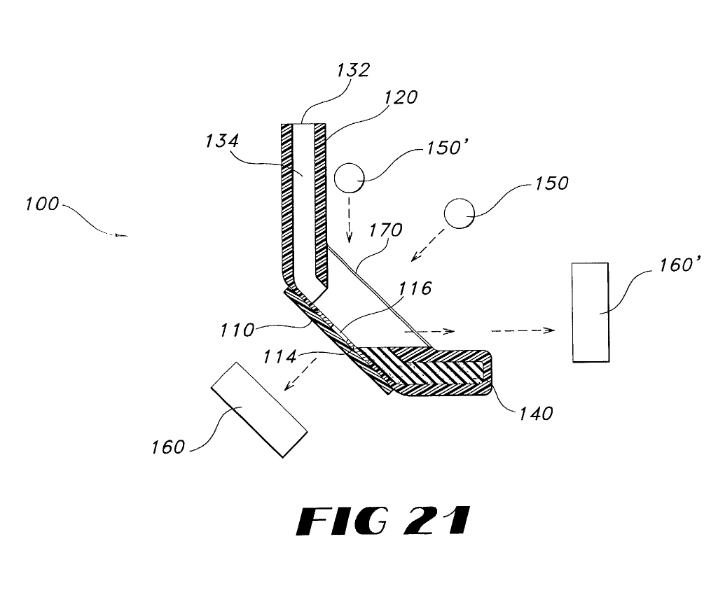

In the examples illustrated in FIGS. 1-4, the diagnostic system is illustrated showing a means 120 for directing a sample to a test surface that is generally perpendicular to the test strip and the binder-printed, test surface. However, means 120 for directing a sample to the diagnostic test surface is not required to be perpendicular to the test surface and may be configured at an angle to the test surface, that is at an angle greater than or less than 90o to the test surface. Examples of such devices are illustrated in FIGS. 9-12 and 17-20. In the examples illustrated in FIGS. 9-12 and 12A, the diagnostic device comprises a means for directing a sample to a test surface 120 that divides a sample into three portions and which directs a portion of the sample to each of the three test loci 115 located on the surface 112 of film 110. In these examples, the means 120 for directing a sample to a test surface further comprises a well 125 for initially receiving a sample. A sample may be deposited into well 125. The well 125 is connected to a plurality of passages, in this example three passages 131 via passage 134. Passage 134 diverges and extends to three passages 131 that then lead to three different test loci 115 so that a sample is divided into three portions and directed to the three test loci 115. The three different test loci 115 may test for three different analytes, for example CRP, IgE and a viral biomarker or indicator; or for two different analytes with one control; three test loci for one analyte or any other combination of analyte(s) and control(s) as may be desired. Devices and systems of the present invention further comprise an additional opening 152 over each test locus 115 so that a light can be directed transmitted through the opening to the test locus and reflected from the binder-printed surface or transmitted through the binder-printed surface. The additional openings are not required and light may be directed through the passages 134 to determine if the test surfaces diffract light. The light source or the passages or openings may be rotated or moved to align the light source with a particular passage or opening. Alternatively, a light source may be split and redirected or multiple light sources may be provided; one light source for each opening.

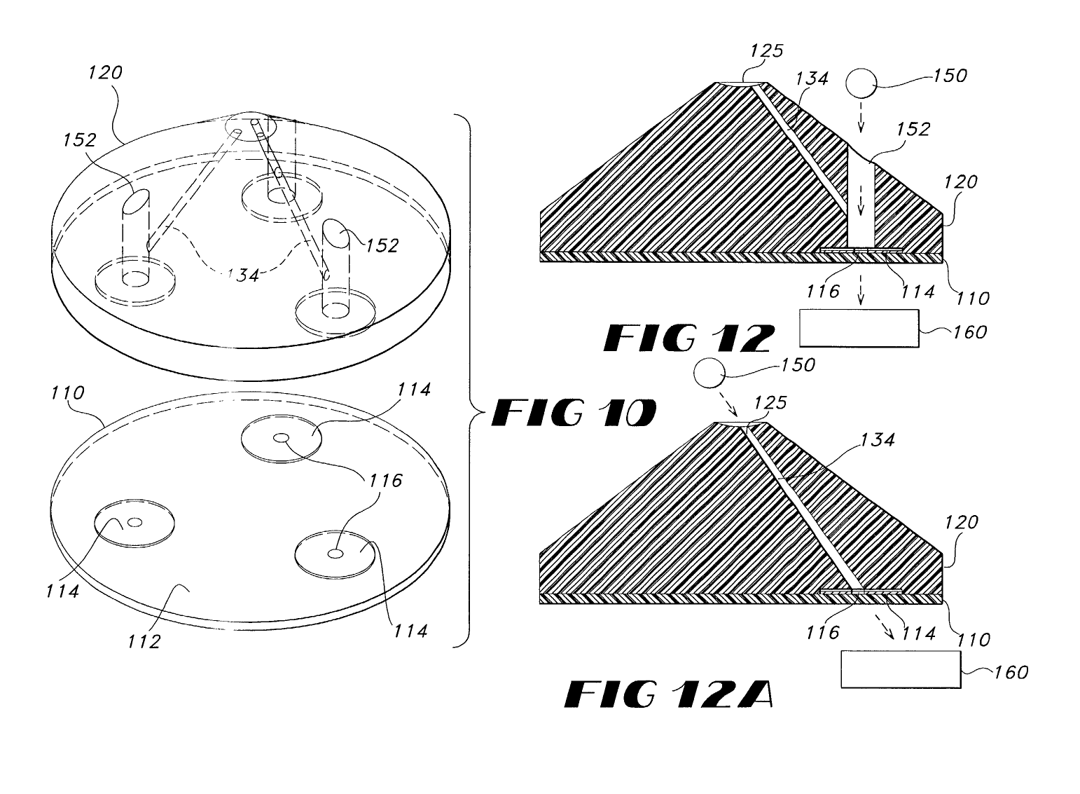

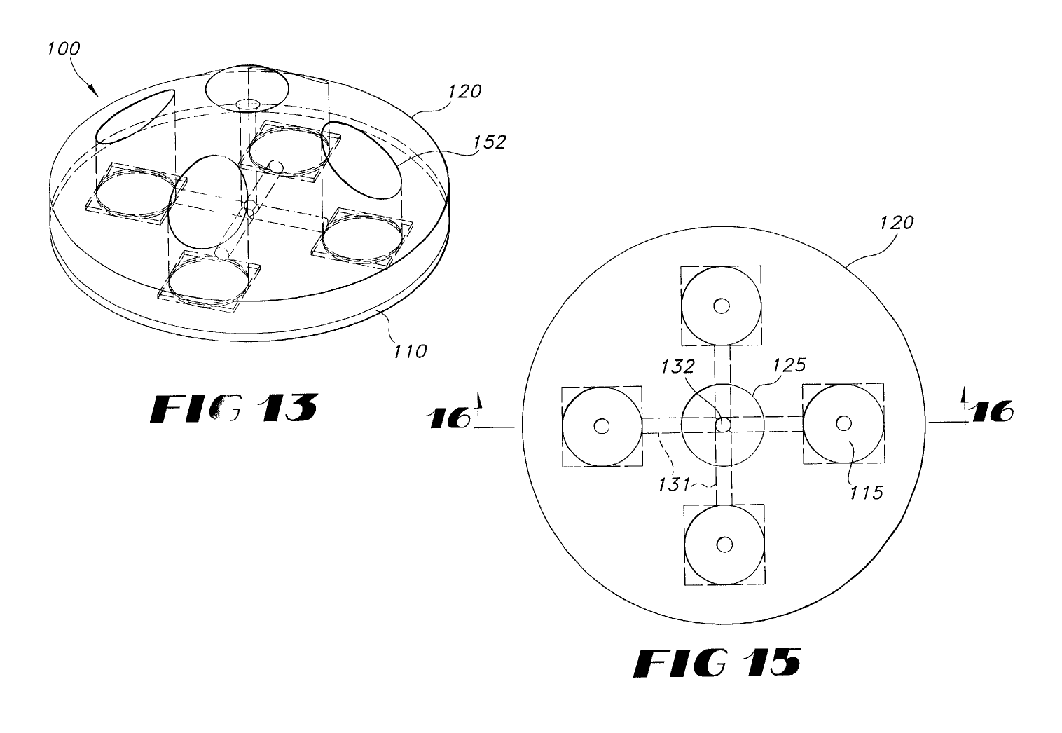

FIGS. 13-16 illustrate yet another example of a device. In this illustrated example, the well 125 extends into conduit 134 that diverges into four conduits 131 at intersection 135 and leads to four different test loci 115 so that a sample is divided into four portions for testing at the four test loci 115. Again, the four different test loci 115 may test for replicate measurements for one analyte, four different analytes, three different analytes at three different test loci with one control test site, two different analytes at two different test loci with two control test loci or otherwise. In an optional desirable embodiment, the device and system of the present invention further comprise an additional opening 152 over each test locus 115 so that light can be directed to and transmitted or reflected through each opening 152.

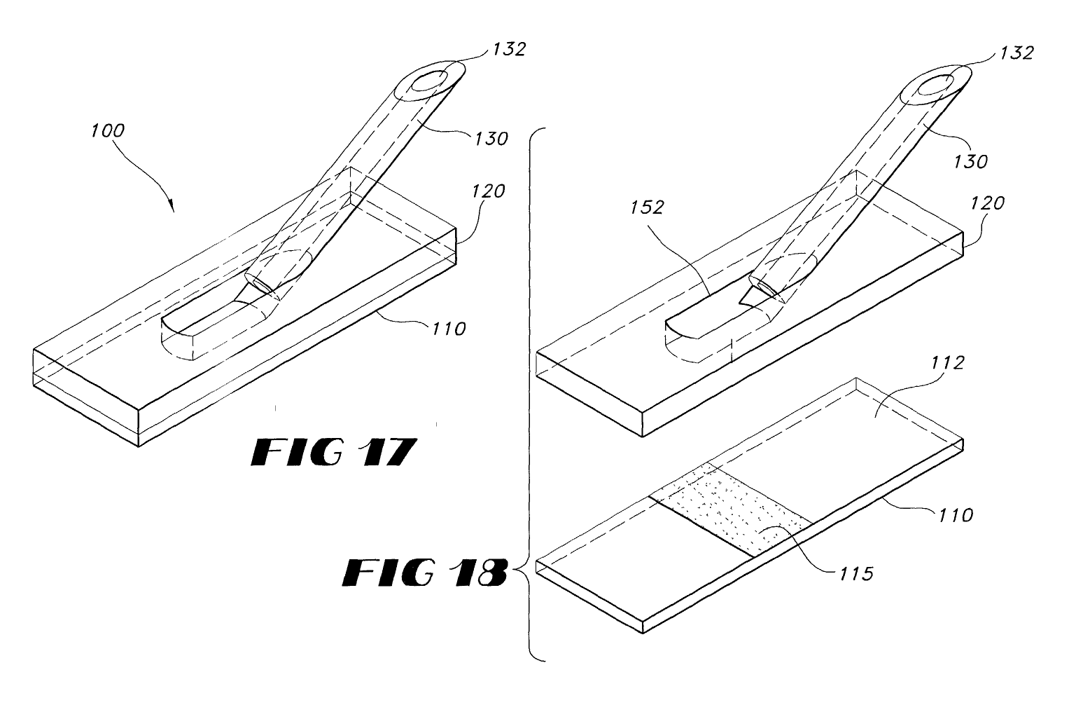

FIGS. 17-20 illustrate yet another example of the present invention. In this illustrated example, the device 100 comprises a means for directing a sample to a test surface 120 that includes a capillary 130 that is angled to facilitate the transmission or reflection of electromagnetic radiation used for detection. The means for directing 120 further includes a first opening 132 that may be beveled to more readily receive a liquid sample. The capillary 130 directs the liquid sample from the beveled opening 132 to the binder-printed surface to test for analyte in the sample. The test surface and device may or may not further include an optional wicking agent layer and/or optional diffraction enhancing elements. The device may also further comprise an opening 152 or a window that transmits light or other electromagnetic radiation through which light may be reflected or transmitted.

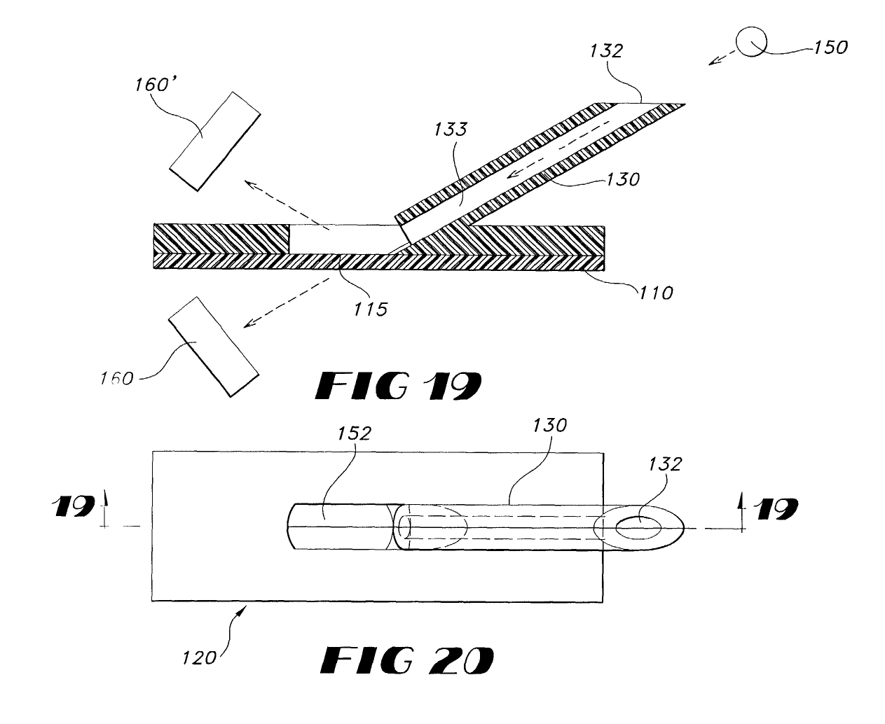

After the sample contacts the test surface and is given sufficient incubation time to bind with the test surface, the presence of binding and the accompanying diffraction can be ascertained via the use of a detector 160 that is positioned to receive and detect radiation that is reflected from the surface of film 110 or transmitted through film 110. The detector 160 may be positioned at the location illustrated at the top of FIG. 19 to receive and detect reflected radiation and at the location illustrated at the bottom of FIG. 19 to receive and detect transmitted radiation. Alternatively, the presence of a diffraction pattern can be ascertained visually by an individual without the use of a detector or an analyzer.

Yet another example of a diagnostic device of the present invention is illustrated in a cross-sectional view in FIG. 21. In the example illustrated in FIG. 21, the diagnostic device 100 includes means 120 for directing a sample to a test surface that further includes an opening 132 for receiving a sample that is in fluid communication with a film 110 upon which a binder is printed via passageway 134. A layer of wicking agent 114 and diffraction enhancing elements may also be included on the binder-printed surface. Optional absorbent material 140 may be provided to aid in directing liquid sample to the binder-printed test surface or to help remove excess sample from the binder-printed surface. The device may also include a window 170 through which light or other electromagnetic radiation may be directed to the binder-printed surface and the layer of wicking agent may also include a hole 116 through which light or other electromagnetic radiation may be directed.

Methods and devices of the present invention may be adapted to detect analytes by methods and devices that are diffraction-based. For example, a device or a method of the present invention may comprise a signal element that will associate with the analyte or a class of analytes that are to be detected. The signal element can be any composition containing any indicator known in the art that provides a detectable and/or measurable manifestation, without a chemical reaction, when the signal element is concentrated in one location. Signal elements can include, but are not limited to, calorimetric compounds, fluorophores, chemo-illuminescent compounds, magnetic compounds, radioactive compounds, compounds that can be detected potentiometrically, light diffraction elements, or combinations thereof. A detectable or measurable manifestation of the signal element can be any means of determining the presence of the element, including but not limited to, any visible means of detection, or any means of detection using devices. Devices for detection include, but are not limited to, counters, spectrophotometers, imaging equipment, magnetic detection devices, radio-activity detection devices, light diffraction measurement devices, potentiometric detection devices, or any combination thereof.

The use of signal elements methods and devices that detect analytes is described in detail in U.S. patent application entitled “Sensors and Methods of Detection for Proteinase Enzymes” and further identified as Express Mail Label EL 602 999 586 US, filed contemporaneously with the present application. For example, the presence of enzymes in humans or animals may be detected using the devices and methods of the present invention. A method for detecting the presence of at least one enzyme in the fluid of a human or animal comprises providing a sample of the fluid and exposing the sample to a signal element and at least one target antibody that is bindable to a target proteinase enzyme to form a target antibody target proteinase enzyme complex. The complex is then exposed to a capture antibody bindable to the target proteinase enzyme in the complex to form a conjugate. The capture antibody is attached to the surface of the reaction site and only complexes that are bound to the capture antibody will be retained in the reaction site. Ideally, capture antibodies for one target proteinase enzyme are placed in each reaction or test site. As the concentration of conjugate increases in the reaction site, it causes a detectable or measurable manifestation due to the concentration of the signal element present in the complex. The identity of the detected target proteinase enzyme can be determined by noting a presence or absence of the detectable or measurable manifestation in a viewing area or by a measurement device. Enzymes can be detected singly or more than one can be detected simultaneously.

While various patents and other reference materials have been incorporated herein by reference, to the extent there is any inconsistency between incorporated material and that of the written specification, the written specification shall control. In addition, while the invention has been described in detail with respect to various specific examples, illustrations and embodiments thereof, it will be apparent to those skilled in the art that various alterations, modifications and other changes may be made to the invention without departing from the spirit and scope of the present invention. It is therefore intended that the appended claims cover all such modifications, alterations and other changes.

### EXAMPLE 1

A gold-coated plastic film (e.g., ˜10 nm thick gold on one side of 3-7 mil MYLAR film sufficient to give <20 ohms/square resistance reading, supplied by CP Films, Inc. of Canoga Park, Calif.) was treated with a blocking agent, beta casein, by soaking the film in a 5 mg/mL solution of beta casein. The solution of beta casein was prepared by dissolving 25 mg of beta casein in 5 mL phosphate buffered saline (PBS) at pH 7.2. After exposure to the beta casein solution for 10 minutes, the film was rinsed with distilled water and dried in an air stream. The treated film, gold-side up, was then contact printed with a thiolated, monoclonal anitibody to C-reactive protein (e.g., Biospacific monoclonal anti-C-reactive protein, Clone#A58040136P) in discrete 10-micron diameter circles on the film to provide a patterned x,y-array of the antibody on the film.

Next, a suspension of antibody-conjugated (Biospacific monoclonal anti-C-reactive protein, Clone#A58110228P) latex microparticles, 0.3 micron diameter at 1.25% solids, was resuspended into a buffer containing 5-10 wt % sucrose and mouse IgG (or optionally, Heterophilic Blocking Reagent, HBR, Cat #3KC534 from Scantibodies of Santee, Calif.). An 11 microliter aliquot of a suspension of antibody-labeled latex microparticles was added by pipetting it on top of the antibody-patterned film. The film with particles was placed in a freezer at ˜−20oC until the particle suspension was frozen (typically >1 hour), and then freeze-dried (˜5-20 mm Hg, using Labconco Model #77500 freeze drying unit with a vacuum pump) to dry the antibody-labeled microparticles on the patterned film surface. A wicking agent (e.g., 0.1 micron pore size Duropore Cat#VVHP04700 from Millipore of Bedford, Mass.) was placed on top of the surface of the microparticle-coated and patterned film (still gold-side up on film). The wicking agent had a 1.6 mm hole cut out of its center (e.g., using a die punch) prior to placing it on the film. This small area (which can range in diameter, for example between 1-3 mm) of the film was not coated with wicking agent to provide a viewing area for diffraction from the sample. The above provided a one-step diagnostic device.

For testing, 34 microliters of sample (e.g., 3.4 μL whole blood with EDTA as anti-coagulant, diluted in 30.6 μL PBS with 0.3% Triton) was added to the top of the film by pipetting this such that the droplet went in the center of the circular area without wicking agent (due to the hole punched out from its center). This caused the blood sample to be slowly, radially wicked away from the gold-coated surface as it was taken in or absorbed by the wicking agent. After the liquid sample had been absorbed by the wicking agent, a clear path for viewing diffraction (or lack thereof) remained through the hole cut from the wicking agent.

Binding was determined microscopically and quantified by determining the percentage within the viewing area (e.g., 1.6 mm) that showed binding of particles in the 10-micron diameter patterned areas. Typically, a 100× magnification was done for this “percent coverage” determination. Also, diffraction was monitored by passing a red helium-neon laser (wavelength 633 nm) through the film.

### EXAMPLE 2

A gold-coated plastic film as described in Example 1, gold-side up, was treated with a blocking agent, e.g., 5 mg/mL beta casein for 10 minutes, rinsed, and dried as described in Example 1. The treated film, gold-side up, was then contact printed with a thiolated, monoclonal antibody to C-reactive protein (e.g., Biospacific monoclonal anti-C-reactive protein, Clone#A58040136P) in 10-micron diameter circles on the film to provide a patterned x,y-array of the antibody on the film. Next, a suspension of antibody-conjugated (e.g., Biospacific monoclonal anti-C-reactive protein, Clone#A58110228P) latex microparticles, 0.3 micron diameter at 1.25% solids, was resuspended into a buffer containing 10% sucrose and mouse IgG (or optionally, Heterophilic Blocking Reagent, HBR, Cat #3KC534 from Scantibodies of Santee, Calif.). A suspension of antibody-labeled latex microparticles was added by pipetting it on top of the antibody-patterned film (typically an aliquot of 4-11 microliters was used of the 1.25% solids conjugated particle sample). The film with particles was frozen and then freeze-dried as described in Example 1. A wicking agent (e.g., 0.6 micron pore size polypropylene, Cat #AN0604700 from Millipore of Bedford, Mass.) was placed on top of the surface of the microparticle-coated and patterned film (still gold-side up on film). The wicking agent had a 1.4 mm hole cut out of its center (e.g., using a die punch) prior to placing it on the film. This small area (which can range in diameter, typically between 1-3 mm) of the film was not coated with wicking agent to provide a viewing area for diffraction from the sample.

This assembly was then placed in a plastic strip housing with capillary tube (e.g., refer to FIGS. 1-4 for exemplary formats) such that there was essentially no gap between the sample film and the capillary tube. Care was taken such that the hole in the wicking agent aligned with the hole in the capillary tube. Also, a hole was cut into the housing of the backing in order to allow a full light path through the capillary tube, through the diffractive film sample, and through the hole placed in the housing.

For testing, 11-34 microliters of the sample was used (e.g., 34 μL of diluted whole blood if 11 microliters particles had been dried on the film surface, or 11 μL diluted blood if only 4 microliters of particles had been dried on the film). The sample was added to the top of the capillary tube tip such that it was pulled into the tube by capillary action and then brought down to the diffraction film surface. The blood sample was then slowly wicked away from the gold-coated surface as it was taken in or absorbed by the wicking agent. After the liquid sample had been absorbed by the wicking agent, diffraction was detected by shining a laser light through the capillary tube such that it was transmitted through the path created by alignment of the holes in the device. A diffraction image was detected. Thus, the sample tested positive for the analyte, CRP in this example.

### EXAMPLE 3

A gold-coated plastic film as described in Example 1, gold-side up, was contact printed with a thiolated, monoclonal anitibody to C-reactive protein (e.g., Biospacific monoclonal anti-C-reactive protein, Clone#A58040136P) in 10-micron diameter circles on the film to provide a patterned x,y-array of the antibody on the film. Next, the printed film was treated with a blocking agent, beta casein, by soaking the film in a 5 mg/mL solution of beta casein. The solution of beta casein was prepared by dissolving 25 mg of beta casein in 5 mL phosphate buffered saline (PBS) at pH 7.2. After exposure to the beta casein solution for 10 minutes, the film was rinsed with distilled water and dried in an air stream.

Next, a suspension of antibody-conjugated (Biospacific monoclonal anti-C-reactive protein, Clone#A58110228P) latex microparticies, 0.3 micron diameter at 1.25% solids, was resuspended into 5% sucrose and HBR reagent (Heterophilic Blocking Reagent, Cat #3KC534 from Scantibodies of Santee, Calif.). The film and 11 microliters of a suspension of antibody-labeled latex microparticles were added by pipetting it on top of the antibody-patterned film. The film with particles was placed in a freezer at ˜−20oC until the particle suspension was frozen (typically >1 hour), and then freeze-dried (˜5-20 mm Hg, using Labconco Model #77500 freeze drying unit with a vacuum pump) to dry the antibody-labeled microparticles on the patterned film surface. A wicking agent (e.g., 0.1 micron pore size Duropore Cat#VWHP04700, from Millipore, Bedford, Mass.) was placed on top of the surface of the microparticle-coated and patterned film (still gold-side up on film). The wicking agent had a 1.6 mm hole cut out of its center (e.g., using a die punch) prior to placing it on the film. This small area (which can range in diameter, typically between 1-3 mm) of the film was not coated with wicking agent to provide a viewing area for diffraction from the sample. The above provided a one-step diagnostic device.

Testing and subsequent measurements were done as described in Example 1. Optionally, testing was done using whole blood that had been diluted with buffer containing 3% Triton X-100. For example, 1.1 microliters of EDTA whole blood was mixed with 9.9 μL of diluent containing 3% Triton; this 11 μL diluted whole blood was added to the top of the film by pipetting it such that the droplet went in the center of the circular area without wicking agent (due to the hole punched out from its center). This caused the blood sample to be slowly, radially wicked away from the gold-coated surface as it was taken in or absorbed by the wicking agent. After the liquid sample had been absorbed by the wicking agent, a clear path for viewing diffraction (or lack thereof) remained through the hole cut from the wicking agent.

We claim:

1. A method for determining the cause of a malady in an animal comprising:
providing a sample from the animal;
providing a test device comprising a plurality of integrated, discrete test sites wherein the plurality of discrete test sites comprises a first discrete test site that comprises a first binder that binds to a first analyte selected from any of groups (i)-(v), wherein group (i) a bacteria or a substance produced by an animal in response to a bacterial infection, group (ii) a virus or a substance produced by an animal in response to a viral infection, group (iii) a fungus and or a substance produced by an animal in response to a fungal infection, group (iv) a protozoa and a substance produced by an animal in response to a protazoan infection and group (v) a substance produced by an animal in response to an allergic reaction, and the plurality of test sites comprises a second discrete test site that comprises a second binder that binds to a second analyte and wherein the second analyte is a different than the first analyte;
introducing the sample to the device wherein the sample contacts the plurality of integrated, discrete test sites;
and evaluating the plurality of integrated, discrete test sites for a change indicating binding of analyte.

2. The method of claim 1, wherein the second analyte that is selected from a different group than the first analyte.

3. The method of claim 2, wherein the first analyte is selected from group (i) and the second analyte selected from group (ii), group (iii), group (iv) or group (v).

4. The method of claim 3, wherein the second analyte is selected from group (v).

5. The method of claim 3, wherein the second analyte is selected from group (iv).

6. The method of claim 3, wherein the second analyte is selected from group (ii).

7. The method of claim 3, wherein the second analyte is selected from group (iii).

8. The method of claim 2, wherein the first analyte is selected from group (ii) and the second analyte is selected from group (iii).

9. The method of claim 2, wherein the first analyte is selected from group (ii) and the second analyte is selected from group (v).

10. The method of claim 2, wherein the first analyte is selected from group (iii) and the second analyte is selected from group (v).

11. The method of claim 1, wherein evaluating the first test site and the second test site for a change indicating that binding has occurred comprises observing light that has been reflected from or transmitted through the first test site for diffraction and observing light that has been reflected from or transmitted through the second test site for diffraction.

12. The method of claim 1, wherein the animal is a mammal.

13. The method of claim 12, wherein the mammal is a human.

14. The method of claim 1, further comprising contacting the sample to a third discrete test site wherein the third discrete test site comprises a third binder that binds to a third analyte selected from any of groups (i)-(v).

15. The method of claim 14, further comprising contacting the sample to a fourth discrete test site wherein the fourth discrete test site comprises a fourth binder that binds a fourth analyte selected from any of groups (i)-(v).

16. The method of claim 1, wherein the test device is a hand-held test device.

17. A diffraction-based diagnostic method for differentiating the causes of a respiratory infection in a human comprising:
providing a fluidic sample from a human;
providing a test device comprising a first discrete test site and a second discrete test site wherein the first discrete test site comprises a first binder that is selected to bind to a first analyte selected from any of groups (i)-(v): group (i) a bacteria or a substance produced by a human in response to a bacterial infection, group (ii) a virus or a substance produced by a human in response to a viral infection, group (iii) a fungi or a substance produced by a human in response to a fungal infection, group (iv) a protozoa or a substance produced by a human in response to a protozoan infection and group (v) an allergen or a substance produced by a human in response to an allergic reaction, and the second discrete test comprises a second binder that binds a second analyte and wherein the second analyte is different than the first analyte;
introducing the sample to the device wherein at least a portion of the sample contacts the first test site and the second test site;
directing light at the first test site and the second test site; and
evaluating light reflected from or transmitted through the first test site for diffraction indicating that binding has occurred at the first test site and evaluating light reflected from or transmitted through the second test site for a change indicating that binding has occurred at the second test site.

18. The method of claim 17, wherein the second analyte is selected from groups (i)-(iv).

19. The method of claim 18, wherein the second analyte is selected from a group that is a different group than the first analyte.

20. The method of claim 19, wherein the first analyte is selected from group (i) and the second analyte is selected from group (ii), group (iii), group (iv) or group (v).

21. The method of claim 20, wherein the second analyte is selected from group (v).

22. The method of claim 20, wherein the first binder is an antibody to C-reactive protein or an antibody to neutrophil lipocalins.

23. The method of claim 21, wherein the second binder is an allergen, an allergen extract or an antibody to IgE.

24. The method of claim 17, wherein the first binder or the second binder is an allergen, an allergen extract or an antibody to IgE.

25. The method of claim 17, wherein the first binder or the second binder is sialic acid or an antibody to influenza.

26. The method of claim 17, wherein the first binder or the second binder is an antibody to Aspergillus.

27. The method of claim 20, wherein the second analyte is selected from group (ii).

28. The method of claim 20, wherein the second analyte is selected from group (iii).

29. The method of claim 19, wherein the first analyte is selected from group (ii) and the second analyte is selected from group (iii).

30. The method of claim 19, wherein the first analyte is selected from group (ii) and the second analyte is selected from group (v).

31. The method of claim 19, wherein the first analyte is selected from group (iii) and the second analyte is selected from group (v).

32. The method of claim 19, further comprising directing a sample to a third discrete test site wherein the third discrete test site comprises a third binder that is adapted to bind to a third analyte selected from any of groups (i)-(v), directing light at the third test site and evaluating light reflected from or transmitted through the third test site for diffraction indicating that a change indicating that binding has occurred at the third test site.

33. The method of claim 32, further comprising directing a sample to a fourth discrete site wherein the fourth discrete test site comprises a fourth binder that is adapted to bind to a fourth analyte selected from any of groups (i)-(v) and evaluating the fourth test site for a change indicating that binding has occurred at the fourth test site.

34. The method of claim 17, wherein the test device is a hand-held device.

35. A device for determining the presence of analytes in a sample comprising:
a surface comprising a first discrete test site wherein the first discrete test site comprises a first binder that is adapted to bind to at least one first analyte selected from any of groups (i)-(v): group (i) a bacteria or a substance produced by an animal in response to a bacterial infection, group (ii) a virus and or a substance produced by an animal in response to a viral infection, group (iii) a fungus or substance s produce by an animal in response to a fungal infection, group (iv) a protozoa or a substance produced by an animal in response to a protozoan infection and group (v) a substance produced by an animal in response to an allergic reaction, and a second discrete test comprising a second binder that is adapted to bind at least one second analyte selected from any of groups (i)-(v) and that is a different binder than the first binder.

36. The device of claim 35, wherein the at least one second analyte that is selected from any of groups (i)-(iv) and is different from the group of the first analyte.

37. The device of claim 36, wherein the first analyte is selected from group (i) and the second analyte is selected from group (ii), group (iii), group (iv) or group (v).

38. The device of claim 37, wherein the second analyte is selected from group (v).

39. The device of claim 37, wherein the first binder is an antibody to C-reactive protein or an antibody to neutrophil lipocalins.

40. The device of claim 38, wherein the second binder is an allergen, an allergen extract or an antibody to IgE.

41. The device of claim 35, wherein the first binder or the second binder is an allergen, an allergen extract or an antibody to IgE.

42. The device of claim 35, wherein the first binder or the second binder is sialic acid or an antibody to influenza.

43. The device of claim 35, wherein the first binder or the second binder is an antibody to Aspergillus.

44. The device of claim 35, further comprising a guide that directs at least a portion of the sample to the first discrete test site and to the second discrete test site.

45. The device of claim 35, further comprising a third discrete test site wherein the third discrete test site comprises a third binder that is adapted to bind to a third analyte and wherein the third analyte is different than both the first analyte and the second analyte.

46. The device of claim 45, further comprising a fourth discrete site wherein the fourth discrete test site comprises a fourth binder that is adapted to bind to a fourth analyte and wherein the fourth analyte is different than the first analyte, the second analyte and the third analyte.

47. The device of claim 35, wherein the device is a hand-held device.

48. The device of claim 35, wherein the first binder is printed in a defined pattern on a substrate and the second binder is printed in a defined pattern on the substrate.

49. The device of claim 35, further comprising diffraction enhancing elements.

50. The device of claim 35, further comprising a wicking agent.

51. A device for determining the cause of a malady in an animal comprising:
a test device comprising discrete first and second test sites and wherein said first and second test sites are integral to said test device,
said first test site comprises a first binder that to binds to an analyte selected from the group consisting of bacteria, substances produced by an animal in response to a bacterial infection, viruses, substances produced by an animal in response to a viral infection, fungi, substances produced by an animal in response to a fungal infection, protozoa, substances produced by an animal in response to a protozoan infection, allergens and substances produced by a animal in response to an allergic reaction; and
said second test site comprises a second binder that binds to a different analyte than the first binder.

52. The device of claim 51 wherein said first binder binds to analytes selected from the group consisting of bacteria and substances produced by an animal in response to a bacterial infection.

53. The device of claim 51 wherein said first binder binds to analytes selected from the group consisting of bacteria and substances produced by an animal in response to a bacterial infection and further wherein said second binder binds to analytes selected from the group consisting of allergens and substances produced by a an animal in response to an allergic reaction.

54. The device of claim 53 wherein the test device further comprises a third test site and wherein said third test site comprises a third binder that binds to analytes selected from the group consisting of viruses and substances produced by an animal in response to a viral infection.

55. The device of claim 51 wherein said first binder binds to analytes selected from the group consisting of bacteria and substances produced by an animal in response to a bacterial infection, and further wherein said second binder binds to analytes selected from the group consisting of viruses and substances produced by an animal in response to a viral infection.

56. The device of claim 51 wherein said first binder binds to analytes selected from the group consisting of bacteria and substances produced by an animal in response to a bacterial infection, and further wherein said second binder binds to analytes selected from the group consisting of fungi and substances produced by an animal in response to a fungal infection.

57. The device of claim 56 wherein the test device further comprises a third test site and wherein said third test site comprises a third binder that binds to analytes selected from the group consisting of protozoa and substances produced by an animal in response to a protozoan infection.

58. The device of claim 51 further comprising a conduit for directing sample to said test sites.

59. The device of claim 58 further comprising a sample port and wherein said sample port is in fluid communication with said conduit.
